(19) United States
(12) Patent Application Publication
(10) Pub. No.: US 2024/0009467 A1
(43) Pub. Date: Jan. 11, 2024

(54) IMPLANTABLE MEDICAL DEVICE, METHOD FOR ATTACHING AN ELECTRODE, A SET AND A USE OF AN IMPLANTABLE MEDICAL DEVICE

(71) Applicant: ZURIMED TECHNOLOGIES AG, Zürich (CH)

(72) Inventors: Elias Bachmann, Zürich (CH); Pol Banzet, Zürich (CH); Xiang Li, Zumikon (CH)

(21) Appl. No.: 18/252,651

(22) PCT Filed: Nov. 12, 2020
(86) PCT No.: PCT/EP2020/081891

§ 371 Date: May 11, 2023

(51) Int. Cl. A61N 1/375

(52) U.S. Cl. CPC A61N 1/37518

(57) ABSTRACT

An implantable medical device including a first patch for electrical stimulation and/or electrical sensing of human or animal soft tissue is disclosed. The first patch includes a felt material. The felt material has a multitude of fibers that are entangled with each other. The felt material is suitable to be felted with fibers of human or animal soft tissue. The first patch includes fibers that are electrically conductive such that the soft tissue can be electrically stimulated and or electrical signals of the soft tissue can be sensed.

### CROSS-REFERENCE TO RELATED APPLICATION(S)

This application is a Section 371 National Stage Entry Application of International Application No. PCT/EP2020/081891, filed Nov. 12, 2020 and published as WO 2022/100834 on May 19, 2022, in English.

### FIELD

The present invention relates to an implantable medical device, a method for attaching a medical device, a set and a use of an implantable medical device according to the preamble of the independent claims.

### BACKGROUND

The field of the present invention relates to the electrical stimulation of human tissue, in particular to providing an implantable medical device that conducts electrical energy to human tissue. There is a variety of applications in which applying electrical energy to human tissue is advantageous. For example, pacemakers are devices that stimulate, or steady the heartbeat or re-establish the rhythm of an arrested heart. The heart is stimulated by sending electrical impulses via electrodes to the heart muscle and the pacemaker includes an electrode that is connected to the heart either internally or via the epicardium.

To remain mechanically attached, electrodes typically have a hook or a screw that is fixated in situ and that damage the tissue during implantation, which scars the surrounding tissue after implantation. Even further, the electrical energy is concentrated at the hook or screw which may cause additional scarring or fibrotic tissue. If extensive formation of fibrotic tissue at the stimulation site occurs, the excitation threshold increases, and the energy efficiency of the pacemaker system is impaired.

WO 2016/044762 Ai describes an apparatus and methods for fabricating tubular structures from a combination of fibrous materials for use in tissue engineering scaffold applications. The fiber scaffolds may include a nonwoven felt that is attached to a further nonwoven felt with an attachment device including a plurality of needles. Thereby, the fibers of the felts are entangled with each other.

Disclosed embodiments of the present invention help to overcome disadvantages of the prior art. In particular, disclosed embodiments of the present invention provide an electrode that transfers electrical energy efficiently into human or animal tissue without damaging or scarring during the transfer of the electrical energy or during implantation. Further, embodiments of the present invention may be additionally providing a secure mechanical attachment of the electrode to the tissue.

### SUMMARY

A first aspect of the invention is directed to an implantable medical device comprising a first patch for electrical stimulation and/or electrical sensing of human or animal soft tissue. The first patch comprises a felt material. The felt material has a multitude of fibers, wherein the fibers are entangled with each other. The felt material is suitable to be felted with fibers of human or animal soft tissue. The first patch comprises fibers that are electrically conductive such that the soft tissue can be electrically stimulated and/or electrical signals of the soft tissue can be sensed.

Thereby, a medical device is provided with fibers that can be interweaved with the fibers of the human or animal tissue. In particular, the fibers of the patch can be pushed or pulled into the soft tissue by means of a needle, pin or blade comprising barbs. The technique of interweaving the fibers with soft tissues is described in the related application PCT/CH2019/000015. The present invention enables a stimulus or signal transmission over a greater area which is particularly advantageous in cases where local stimuli are insufficient. By interweaving electrically conductive fibers with soft human animal tissue, an electrode and an interface are provided with a low electrical impedance that prevents scarring of the tissue and prevents fibrosis. At the same time, a strong mechanical connection between the patch and the soft tissue is provided. It is believed, without being limited to this, that the resulting larger surface area between the electrically conductive fibers and the soft tissue is responsible for a lower impedance in the interface between the electrically conductive fibers and the soft tissue.

In a preferred embodiment, the electrically conductive fibers are entangled with the multitude of fibers. Thereby, a single patch with electrically conductive properties is provided, that can be felted to human or animal soft tissue. Alternatively, such a single patch with conductive properties may be provided during implantation as a result of the felting. Further, the mechanical connection and the electrical connection is simultaneously established during the felting. In some embodiments, the multitude of fibers of the felt material may form the electrically conductive fibers. In other embodiments, the multitude of fibers that are entangled may comprise conductive and nonconductive fibers. Additionally or alternatively, conductive fibers may be directly felted to the soft tissue.

The entangled multitude of fibers may be porous. The patch may have a volume of 0.5-600 mm³, and in some embodiments 1.7-120 mm³. The low volume increases the current density for a same current as compared to a solid patch. An increased current density may lower the voltage threshold for polarization. An electrode with a low volume due to the porosity increases the current density in the electrode (i.e. in the conductive fibers), which may lower the voltage threshold for polarization and the electrical resistance. The entangled fibers thus create a situation similar to a porous material, where the ratio of surface to volume of matter is higher than for a purely non-porous solid material such as a sphere (minimal ratio of surface to volume). Further, the high current density and the relatively high surface contact area results in a low impedance at the electrode-tissue interface, which can be beneficial to reduce the after-potential voltage, which can hinder proper function of the pacemaker. Fibrous (and porous) electrodes have both a small overall volume, and a high contact surface due to their geometry.

The fibers may be absorbable or non-absorbable. In certain embodiments the fibers comprise or consist of non-absorbable fibers, particularly silk, polypropylene, polyester (e.g. PET, CAS No. 25038-59-9), polytetrafluorethylene (e.g. PTFE, CAS No. 9002-84-0), nylon or a polyamide. Herein, the term non-absorbable may relate to materials which are not degraded after implantation in a human or animal body.

In certain embodiments, the medical implant comprises or consists of absorbable fibers, particularly polyglycolic acid (PGA, CAS No. 26124-68-5), polylactic acid (PLGA, CAS No. 26780-50-7 and PLLA, CAS No. 33135-50-1), polydioxanone (PDO, CAS 57-55-6), or caprolactone (PCL, CAS No. 24980-41-4). Herein, the term absorbable may relate to materials which are degraded after implantation in a human or animal body. Such fibers could initially provide necessary mechanical anchoring, and then get absorbed as the body encapsulates the conductive fibers, rendering the mechanical fibers dispensable. In certain embodiments, the medical implant may comprise or consist of a combination of absorbable fibers and non-absorbable fibers.

In a preferred embodiment, the electrically conductive fibers are made of or comprise a metal. Preferred metals are steel, in particular stainless steel, i.e. steel containing at least 10% chromium (e.g. 316 L with 17 to 19 w % Cr and 13 to 15 w % Ni), titanium, platinum, platinum-iridium, platinized titanium coated platinum, iridium oxide, magnesium, gold and silver, or any alloy thereof.

In a preferred embodiment the fibers are tear-resistant. Tear-resistant may be understood as a sufficient tensile strength, fracture strength and flexibility such that the fibers will not tear or break when they are felted, e.g. felted as described in PCT/CH2019/000015.

In a particularly preferred embodiment, the electrically conductive fibers and/or the nonconductive fibers have a yield strength that is higher than a maximum stress during felting.

The electrically conductive and nonconductive fibers are biocompatible and preferably ductile, i.e. not too brittle. This may mean that the maximum stress to which the fibers are typically exposed during felting is less than the yield stress of the fibers (σ_{yield}<σₘₐₓ). The maximum stress to which a fiber may be exposed during felting may be understood by the following formula:

$${σ}_{max} = \frac{3 * D * E * r}{L}$$

wherein σₘₐₓ is maximum stress to which a fiber is exposed to during felting, D is the deflection of the fiber (i.e. the distance the fiber is carried by the felting needle), E is Young's modulus of the fiber material, r is the radius of the fiber and L is the length of the fiber. The deflection in the present felting of the patch to soft tissue is typically 25 mm or less, but may also be 20 mm or less, 16 or less mm, or 12 mm or less.

In some embodiment the yield strength of the fibers is at least 120 MPa. As can be seen from the above formula, the yield strength may depend on the chosen material and the properties and measurements of the fibers.

The maximum stress of the electrically conductive and/or nonconductive fibers is preferably lower than the yield stress of the fiber. Thereby, a breaking of the fibers is prevented during felting.

Preferably, the nonconductive fibers are stronger than the conductive fibers. The nonconductive fibers may have a higher Young's modulus. Preferably though the nonconductive fibers are able to withstand a higher tensile force.

A ratio between the tensile force exertable on the electrically nonconductive fibers and on the conductive fibers of the felt material is minimum 1.5, preferably minimum 2.0. Therewith the advantage can be achieved that by means of the additional fibers of higher tensile strength or having a larger diameter a higher tensile force can be applied to the felted patch or matting so as to significantly improve the stiffness and patch stability. In a further preferred embodiment, conductive fibers are provided by coating the above mentioned, nonconductive fibers with a conductive material.

In another embodiment the fibers of the felt material are aligned. Thereby, higher unidirectional or multidirectional stiffness properties of the patch be achieved as compared to a common felt material with randomly arranged fibers.

In another embodiment the conductive fibers of the felt material are aligned. This has the advantage that the electrical conductivity of the felt patch can be adjusted to the intended flow of energy, e.g. from the wire to the attachment surface.

In a preferred embodiment, the electrically conductive fibers comprise a biocompatible electroconductive coating. In particular, only a part of the fibers comprises the electroconductive coating or all of the fibers comprise the electroconductive coating. Examples for fibers with a coating are platinum-coated polyester fibers, platinum-coated titanium fibers, iridium oxide-coated cotton fibers, gold-coated polyethylene (PE) fibers, silver-coated polyamide fibers, and silver-coated nylon fibers.

In a preferred embodiment, 50 to 90% of the fibers are nonconductive. 10 to 50% of the fibers may be conductive. Thereby, a balance can be struck between a sufficient mechanical attachment and sufficient electrical conductivity.

In a preferred embodiment, the electrically conductive fibers are interweaved to form a felt metallic wool.

In a preferred embodiment, the first patch has an electrical impedance between 10 and 20,000 Ohm, further preferred between 100 and 10,000 Ohm, particularly preferred between 200 and 2,000 Ohm. The electrical impedance may be measured across an attachment surface for the soft tissue and an interface for an electrical connection (e.g. an electrical wire). The electrical impedance values may refer to a patch that includes 10 to 1000, preferably 50 to 200, felted fibers or 10 to 1000, preferably 50 to 200 needle penetrations.

The patch may be suitable for an energy delivery of 0.1-100 mJ, preferably 1-40 mJ, most preferably 10-20 μJ. The patch may also be suitable for an attachment force of a patch-tissue connection: 1 to 100 N, preferably 5 to 25 N, most preferably 7 to 12 N.

The electrically conductive fibers may have a thickness between 0.2 and 70 μm, preferably between 1 and 50 μm and most preferred between 5 and 10 μm. The electrically conductive fibers may have a length between 10 and 100 mm, preferably between 20 and 60 mm, most preferably between 40 and 50 mm. These properties (alone or in combination) may render the electrically conductive fibers suitable to be felted while retaining their electrical properties and being sufficiently flexible to be pushed or pulled by a needle during felting.

The felts may have a thickness between 0.1 and 4 mm, preferably between 1 and 3 mm, most preferably a thickness of 2 mm.

In certain embodiments, the first (and/or second) patch comprises a stiffness of 30 N/mm to 300 N/mm, particularly 60 N/mm to 250 N/mm, more particularly 130 N/mm to 220 N/mm. Therein the term “stiffness” when relating to the medical device designates a force required per unit of length of elongation of the medical device when the medical textile and the medical implant, particularly the suture, (or medical implants in case of more than one medical implant) are pulled apart.

In a further embodiment, the patch comprises electrically conductive fibers and electrically nonconductive fibers.

In a preferred embodiment the patch comprises an attachment surface. The electrically conductive fibers may be distributed over at least 80% of the entire area of the attachment surface, preferably at least 90% most preferably at least 95%. The attachment surface may be understood as the area of the patch that is intended to be felted to the soft tissue. In some embodiments the fibers are evenly or unevenly distributed over the area of the attachment surface.

The electrically conductive fibers may have a higher concentration at the connection interface for a wire than on an outer edge of the patch.

In a preferred embodiment, the device additionally comprises a wire, preferably an insulated wire, wherein the wire is electrically connected to the first patch. Thereby, the electrical energy may be supplied to the patch and/or electrical signals detected by the patch may be conducted.

In a preferred embodiment, the wire comprises strands, wherein the strands of the wire are electrically conductively connected to the patch. For example, the strands may be felted to the first patch. Alternatively, the wire can be soldered or snapped or riveted to the patch. In one particular embodiment, the strands of the wires may form the electrically conductive fibers.

In a preferred embodiment, the device comprises a second patch comprising a felt material having a multitude of fibers. The fibers of the second patch are entangled with each other and the felt material is suitable to be felted with the fibers of the human or animal soft tissue. The first and second patch may be stacked and the first and second patch may be felted together. Thereby, a secure attachment of the medical device is secured while at the same time providing a low impedance interface.

In a preferred embodiment, the device comprises a second patch comprising a felt material having a multitude of fibers, wherein the fibers are entangled with each other. The felt material is suitable to be felted with fibers of human or animal soft tissue and the second patch is dimensioned such that the first patch can be fixated to the soft tissue but felting the second patch to the soft tissue. Thereby, a secure connection between the first and second patch is ensured.

A further aspect of the invention is directed to a method for manufacturing an implantable medical device, preferably an implantable medical device as described above. The felt with conductive fibers may be manufactured by embedding conductive fibers into a nonconductive felt by needle felting. The conductive fibers may be nonwoven, and may be aligned and spread, e.g. by using a fine comb-like tool. Then the conductive wires may be embedded into the nonconductive felt using a felting needle (or an array thereof). The felting needle may be a needle with notches designed to grab on loose fibers and push them into felt.

Another aspect of the invention relates to a method for attaching an electrode to human or animal soft tissue. The method comprises the step of providing a medical device as described above and felting an attachment surface of the patch of the medical device to the human or animal soft tissue with at least one felting needle. The method may include moving the at least one felting through the patch as described e.g. in PCT/CH2019/000015.

A further aspect of the invention relates to a set comprising a medical device as described above and a deployment device for the implantable medical device. The deployment device may be minimally invasive and/or may be adapted to felt the medical device to the soft tissue, preferably by using at least one felting needle that can be moved reciprocally.

A further aspect of the invention relates to the use of an implantable medical device as described above to treat sinus syndrome, atrial fibrillation, a heart block such as a sinoatrial node block, an atrioventricular node block or infra-hisian block or a neurological disorder such as a spinal cord injury or a stroke.

The medical device may form an electrode and may be used in these applications to conduct an electrical stimulation, for example to the heart muscle for pacing. The medical device may for example be attached inside one of the chambers or the atria of the heart or at the epicardium. Another application may include an electrical stimulation of any muscle or nerve of the body or the sensing (i.e. detection) of an excitation of muscle or nerve bundles. Feltable electrodes can also be attached to other areas such as the skin by felting.

The medical device may be combined with or connected to a pulse generator for the stimulation or may be combined with or connected to circuitry for evaluating the excitation sensed by the medical device. The medical device may be combined with a device such as a pulse generator or evaluation circuitry or be provided independently therefrom. In particular, the pulse generator or evaluation circuitry may be connected to the above-mentioned wire.

The present summary is provided only by way of example and not limitation. Other aspects of the present invention will be appreciated in view of the entirety of the present disclosure, including the entire text, claims, and accompanying figures.

### BRIEF DESCRIPTION OF THE DRAWINGS

Non-limiting embodiments of the invention are described, by way of example only, with respect to the accompanying drawings, in which:

FIG. 1: shows a perspective view of a first embodiment of an implantable medical device according to the invention.

FIG. 2: shows a perspective view of a second embodiment of an implantable medical device according to the invention in an exploded view.

FIG. 3: shows a cross-sectional view of the second embodiment when implanted and felted to soft tissue.

FIG. 4: shows a third embodiment of an implantable medical device according to the invention prior to felting the medical device to the soft tissue.

FIG. 5: shows a minimally invasive deployment device for the implantable medical device in a perspective view.

FIG. 6: shows a cross-sectional view of the deployment device of FIG. 5 with an implantable medical device prior to deployment of the medical device.

FIG. 7: shows a cross-sectional view of the deployment device of FIGS. 5 and 6 with an implantable medical device during to deployment of the medical device.

FIG. 8: show a second embodiment of a deployment device for the implantable medical device in a perspective view.

While the above-identified figures set forth one or more embodiments of the present invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale, and applications and embodiments of the present invention may include features, steps, and/or components not specifically shown in the drawings.

### DETAILED DESCRIPTION OF ILLUSTRATIVE EMBODIMENTS

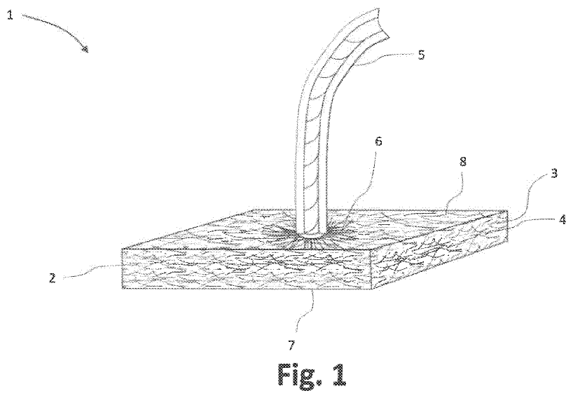

FIG. 1 shows a perspective view of a first embodiment of an implantable medical device 1 according to the invention. The device 1 includes a patch 2. The patch 2 can be provided by coating the fibers of a synthetic or natural nonconductive felt patch with a conductive material. In one example the felt may be dip-coated or spray coated or sputtered with the conductive material.

Preferably, the patch 2 is provided by creating a felted patch of made of a mix of biocompatible, ductile nonconductive (e.g. nylon or cotton) and conductive (e.g. stainless steel, platinum-iridium) fibers 3, 4. The conductive and nonconductive fibers 3, 4 may be stacked on top of each other and punctured with numerous needles that are barbed multiple times. Through repeated puncturing with barbed needles, the fibers are intertwined and/or pressed into the felt. The resulting mixed felt combines the conductivity of feltable conductive fibres and the mechanical strength and durability of nonconductive fibers felt.

The device comprises an insulated lead wire 5. One end of the wire is connected to an implantable pulse generator port (e.g. IS-1, DF-1), and while the other end may be uninsulated. The conductive lead wire can be a coiled monofilament, solid core, or multiple wires braided together. The opposing end is electrically connected to the patch 2. In one embodiment the strands of the wire are uninsulated and form a frayed end 6 as shown in in FIG. 1. The frayed end 5 is felted to the patch 2, thereby connecting the wire electrically and mechanically to the electrically conductive fibers 3 of the patch 2. Alternatively, the end of the wire may be anchored using an additional mechanical attachment such as riveting it into the patch.

The strands of the frayed end should be enough of them, and they should be flexible and stiff enough to be felted (i.e. thin enough while being stiff enough). Suitable dimensions for the wire as well as suitable materials may be similarly selected as described above for the fibers. In some embodiments the diameter of frayed end fibers should be in the range of 0.05 to 50 μm, preferably 0.5 to 5 μm to minimize risk of breakage during felting.

The patch 2 includes an attachment surface 7. The attachment surface 7 is directed towards a soft tissue. In order to attach the patch to the soft tissue, a felting needle is pushed through surface 8 through the patch 2. The felting needle includes barbs that pull the fibers 3, 4 through the attachment surface and into the soft tissue. By repeating this process multiple times, multiple electrically conductive fibers are pushed into the soft tissue and create an electrical connection between the patch and the soft tissue.

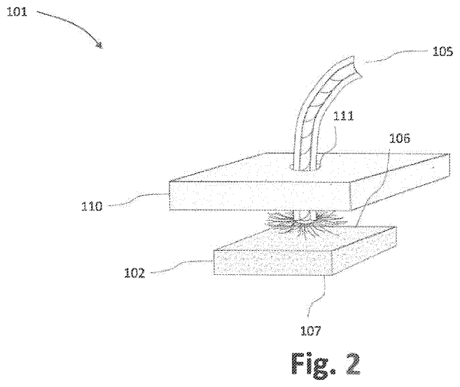

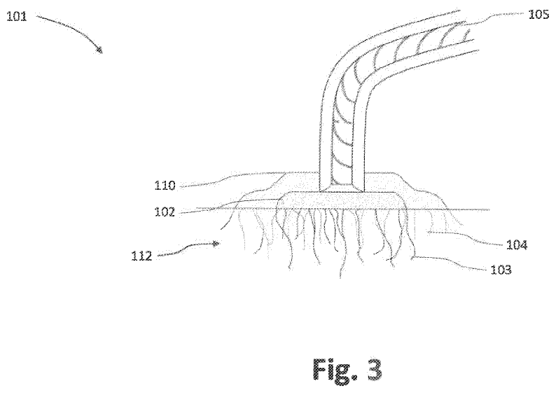

FIGS. 2 and 3 show a second embodiment of an implantable medical device 101 according to the invention. The implantable medical device 101 includes a first patch 102 and a second patch 110. In one embodiment, the first patch 102 may be similar to patch 2 as shown above. In another embodiment, the first patch 102 is a conductive felt patch made of biocompatible conductive fibers. The first patch 102 may be partially or entirely of conductive or conductively coated fibres. A lead wire 105 is electrically connected to the first patch similar to lead wire 5.

In addition, the medical device comprises the second patch 110. The second patch 110 is a felt patch made of biocompatible nonconductive fibers, synthetic (e.g. PTFE, PET) or natural (e.g. silk). The second patch 110 is includes a hole 111. The lead wire is guided through the hole 111.

As shown in FIGS. 2 and 3, the patches 102 and 110 are stacked with the conductive material being arranged towards soft tissue 112 (i.e. at the bottom in the schematic drawings of FIGS. 2 and 3). The second, nonconductive patch 110 is arranged parallel to the first patch 102 and on top of the first patch 102. The frayed end 106 of the wire 105 is arranged in between the two patches 102, 110. The conductive wire 105 is fed through the hole 111 of the nonconductive patch. The device is then pre-felted together. Thereafter, the device is positioned, and then felted to soft tissue, e.g. muscle tissue, using the felting technique as described in PCT/CH2019/000015. Alternatively, the pre-felting step may be omitted and the patches 102, 110 and the frayed end are stacked onto each other and are then directly felted to each and to the soft tissue (e.g. muscle tissue).

The nonconductive patch 110 provides additional structural integrity and strong anchoring to the tissue. Further, the nonconductive patch may isolate the conductive patch on one side, preventing power losses during stimulation and/or interfering signals during sensing. In certain embodiments, the nonconductive patch 110 is connected to the insulation layer of the lead wire 105 so that loads (e.g. pulling force on the wire 105) can be transferred to both patches 105, 106. The wire 105 is felted to the conductive patch 102. The conductive patch 110 is felted to the tissue, which ensures electrical conductivity between the tissue and the implantable pulse generator.

As can be seen from FIG. 3, due to the felting, the conductive fibers 103 are pushed into the soft tissue 112 resulting in a low impedance of the interface between the wire 105 and the soft tissue 112. Further, the nonconductive fibers 104 are also pushed into the soft tissue 112, which provides a sufficiently strong mechanical connection.

The electrical and mechanical connection between the electrical wire and conductive felt can also be achieved through other means than felting its frayed ends. The wire can be directly soldered to the conductive patch. A snap or rivet connection can also be used.

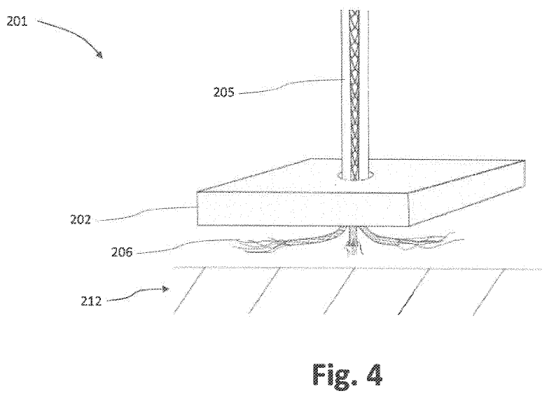

FIG. 4 shows a third embodiment of an implantable medical device 201 according to the invention prior to felting the medical device to the soft tissue. In this embodiment the multifilament or braided conductive wire 205 with frayed ends 206 is directly used for both mechanical bond and electrical conduction, without the need for any additional patch. The dense frayed wire of biocompatible conductive filaments is directly felted into the soft tissue 212.

If needed, for additional mechanical support, a nonconductive felt patch can be added. This process is similar to the stacked method previously described with reference to FIGS. 2 and 3.

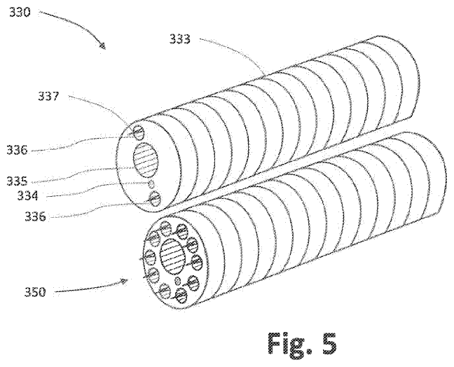

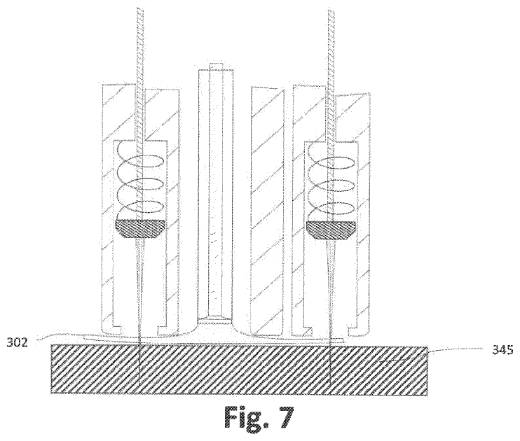

FIGS. 5 to 7 show a minimally invasive deployment devices 330 and 350 for any of the implantable medical devices 1, 101, 201 shown above. During deployment, a guidewire is first introduced through an access sheath into the desired vessel and delivered to the target zone. Then, the minimally invasive deployment device 330 or the minimally invasive deployment device 350 is passed over the guidewire to the right position.

The minimally invasive deployment device 330 is formed by a catheter 333, that may be flexible. The flexible catheter 333 includes a guidewire lumen 334. The guidewire lumen is slid over the previously inserted guidewire and allows the device 330 to be brought to a target site. In addition, the catheter 333 includes an implant lumen 335. The medical implant 301 is held in the implant lumen 335. Further, the catheter 333 includes a felting needle mechanism, that is arranged at the distal end of the catheter 333. As can be seen from FIG. 5, the catheter includes two further needle lumens 336, within each of which a felting needle 37 is arranged. The minimally invasive deployment devices 330 and 350 are only different from each other in that the felting needle mechanism of the minimally invasive deployment device 350 includes ten felting needles 337 with ten corresponding needle lumens 337 instead of two felting needles 337 with two corresponding needle lumens 337.

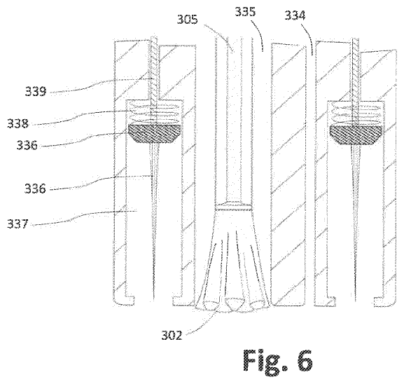

FIGS. 5 and 6 show a cross section of the minimally invasive deployment device 330 (though it could also be a cross-section of the minimally invasive deployment device 350). The felting needle mechanism can be understood from the cross-sections in FIGS. 5 and 6 and includes the needle lumen 337 and the felting needle 336. The felting needle 336 is pushed in a distal direction by a spring 338 and a spring stopper 339. The needle 336 may be pulled in a proximal direction by a cord 339. Thus, the felting needle can be moved back and forth with a reciprocal motion by pulling and releasing the cord 339. The needle 337 may have a length of length from 5 to 30 mm and may include barbs (not shown).

In the implant lumen 335 (i.e. the central lumen), a folded feltable medical device 301 is arranged. The feltable medical device 301 may be an implantable medical device as shown in one of the previous figures. The implantable medical device includes a folded patch 302 and a wire 305 attached to the patch. The folded patch 302 may include a folded self-expanding outer ring (made of e.g. Nitinol, not shown), to ensure proper deployment of the patch of the medical device, when it is pushed out of the implant lumen 335. The patch 302 can be pushed out of the sheath manually, e.g. by pushing the wire 305. Thereby, the patch 302 is deployed. Then, the catheter 333 is pushed in its distal direction, which presses the patch 302 against the soft tissue 345. Then, the needling mechanism described above is activated, felting the electrode into the tissue. Once a satisfactory electrical and mechanical connection is achieved, the sheath can be retracted out of the body. The lead wire is left in place and can then be connected to an implantable pulse generator such as a pacemaker.

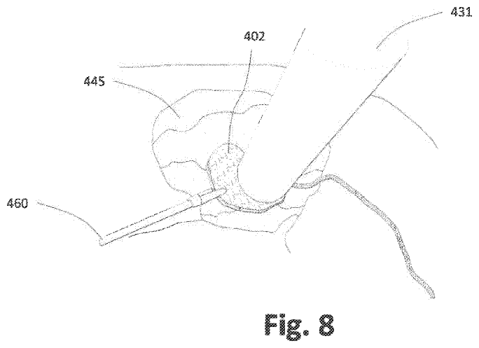

FIG. 8 shows a second embodiment of a deployment device for the implantable medical device in a perspective view. If the implantation is in a less space constrained implantation, separate devices for the deployment of the implantable medical device 401 and for the felting can be used. Such a case could be, for example, anchoring a pericardial electrode using video assisted thoracotomy, to provide left ventricular pacing for cardiac resynchronization therapy. Using surgical tools, a medical device 401 such as described above is held against the desired stimulation or sensing location. The implantable medical device 401 may be deployed with a clamp or grasper 460. Then, the patch 402 (or the patches, if more than one is used) are felted to the soft tissue 445 below with a felting device 431. The felting device may comprise a needling mechanism as described with respect to FIGS. 5 and 6, which secures the lead mechanically and ensures a good electrical conduction.

Although the present invention has been described with reference to preferred embodiments, workers skilled in the art will recognize that changes may be made in form and detail without departing from the spirit and scope of the invention.

1. An implantable medical device comprising a first patch for electrical stimulation and/or electrical sensing of human or animal soft tissue, the first patch comprising:
a felt material having a multitude of fibers, wherein the fibers are entangled with each other, and wherein the felt material is suitable to be felted with fibers of the human or animal soft tissue,
wherein the first patch further includes fibers that are electrically conductive such that the soft tissue can be electrically stimulated and/or electrical signals of the soft tissue can be sensed.

2. The device according to claim 1, wherein the electrically conductive fibers are entangled with the multitude of fibers.

3. The device according to claim 1, wherein the electrically conductive fibers comprise or are made of a metal.

4. The device according to claim 1, wherein the electrically conductive fibers comprise a biocompatible electroconductive coating.

5. The device according to claim 1, wherein the electrically conductive fibers are interweaved to form a feltable metallic wool.

6. (canceled)

7. The device according to claim 1, wherein the electrically conductive fibers and/or nonconductive fibers of the multitude of fibers have a yield strength that is higher than a maximum stress during felting.

8. The device according to claim 1, wherein the patch has an electrical impedance between 10 and 20,000 Ohm.

9. (canceled)

10. The device according to claim 1, wherein the patch comprises an attachment surface, wherein the electrically conductive fibers are distributed over at least 80% of an entire area of the attachment surface.

11. The device according to claim 1, wherein the device additionally comprises:
a wire, wherein the wire is electrically connected to the first patch.

12. The device according to claim 11, wherein the wire comprises strands and wherein the strands are felted to the first patch.

13-15. (canceled)

16. A set comprising a medical device of claim 1, and a deployment device for the implantable medical device.

17. (canceled)

18. The device according to claim 11, wherein the wire comprises an insulated wire.
